(19) United States
(12) Patent Application Publication
(10) Pub. No.: US 2026/0256308 A1
(43) Pub. Date: Sep. 3, 2026

(54) Wireless electronic smart drinking straw tip hydration system.

(71) Applicant: Nayland Glaznieks, Nuneaton (GB)

(72) Inventor: Nayland Glaznieks, Nuneaton (GB)

(21) Appl. No.: 19/067,924

(22) Filed: Mar. 2, 2025

(30) Foreign Application Priority Data
Jan. 7, 2024 (GB) GB2400209.9

(51) Int. Cl. A47G 21/18; A61B 5/00

(52) U.S. Cl. CPC A47G 21/189; A61B 5/4875; A61B 5/6887; A61B 5/7246; A61B 2560/0257; A61B 2562/0219

(57) ABSTRACT

A wireless electronic smart drinking straw tip hydration system that monitors a user's hydration levels and prompts user to drink if below a recommended target consumption level.

Sensors inside the smart drinking straw tip assembly measure the user's liquid consumption and send the consumption data to a custom software application installed on a wireless device. The custom software application will inform the user how much liquid they have consumed through the smart drinking straw tip assembly. Based on users details, the custom software application recommends a daily liquid target consumption and compares it to the actual consumption through the smart drinking straw tip assembly. If the consumption rate is below the recommended scheduled liquid consumption, the custom software application will send a signal via the wireless device to the smart drinking straw tip assembly which gives a visual or sound indication to the user, prompting them to drink.

This invention relates to a wireless electronic smart drinking straw tip hydration system. The system is made up of an wireless electronic smart drinking straw tip assembly and custom software application installed on a wireless device (e.g) smart phone.

Keeping hydrated is critical for physical and mental health but many people do not consume enough fluids each day. Around 60% of the human body is made up of liquid and around 90% of human blood is liquid. Dehydration can cause the blood to become thick and increase blood pressure increasing the risk of heart related issues, it can also affect brain function, leading to mental health issues.

Given the importance of human body hydration it is beneficial to track hydration levels and increase levels where users are not sufficiently hydrated.

There are already smart liquid bottles that tracker consumption by measuring the amount of liquid inside the bottle. These require the person to always use the same bottle to track consumption, which is not practical, especially when in multiple locations a day. To overcome this problem, the present invention proposes using a smart drinking straw tip assembly which can be fitted to any drinking straw and carried to multiple locations.

A Wireless electronic smart drinking straw tip hydration system that monitors the user's hydration levels by calculating liquid consumption through a smart drinking straw tip assembly by using a pressure sensor, orientation sensor and non-contact liquid detection sensor.

The smart drinking straw tip assembly then calculates the liquid consumed and sends the amount to the custom software application installed on a wireless smart device (e.g) Smart phone. Based on users' details, the custom software application recommends a target consumption schedule which it compares to the actual liquid consumed via the smart drinking straw tip assembly. If the actual liquid consumed is less than recommended target scheduled consumption amount the custom software software application will then calculate current consumption rate and check time since last indication to user before determining if an indication to user is needed. If needed the custom software application sends a signal back to the smart drinking straw tip assembly which triggers an audio or visual indicator on the smart drinking straw tip assembly to prompt the user to drink more.

The recommended target consumption on the custom software application is primary based on user inputs age, weight and gender. The recommended target consumption is also adjusted for activity level of user (e.g) from an activity tracker software application installed on the same wireless device and humidity/temperature of users' location from weather software application installed on the same wireless device.

There are factors that effect on users' consumption through a drinking straw which the three sensors measure.
1) Pressure difference between the pressure inside smart drinking straw tip assembly and atmospheric pressure acting on the surface of the liquid in the container—obtained from the pressure sensor—see FIG. 4 for more details.
2) Orientation of the smart drinking straw tip assembly—obtained from orientation sensor see FIG. 5 for more details. When the straw is in the upright position user's actual consumption can be accurately estimated without the orientation sensor data. Orientation sensor data is needed to increase accuracy when the straw is not upright (at an angle).
3) Pressure difference when non-contact liquid detection sensor initially detects liquid inside smart drinking straw tip assembly hollow tube and current pressure difference. By knowing when liquid has reached the smart drinking straw tip assembly via the non-contact liquid sensor, we can use the pressure difference to compare against current pressure difference to calculate users' actual liquid consumption.
For consumption to occur the current pressure difference must be greater than the pressure difference to get liquid up into the drinking straw tip and detected by the non-contact liquid detection sensor. See FIG. 6 for more details.

The invention will now be described solely by way of example and with reference to accompanying figures:—

### LIST OF FIGURES

FIG. 1A shows a summary view of the invented components (100,200) and support components of the wireless electronic smart drinking straw tip hydration system (001).

FIG. 1B shows the network diagram of component connections that make up the wireless electronic smart drinking straw tip hydration system (001).

FIG. 2A shows the physical representation of the smart drinking straw tip assembly (100) & components (101 to 114).

FIG. 2B—Example screenshot of the custom software application (200) & components (250 to 260)

FIG. 3A shows a flow diagram of the smart drinking straw tip assembly (100) calculation.

FIG. 3B shows a flow diagram for the custom software application (200) installed on a smart wireless device (400).

FIG. 4 shows different pressure differences between inside smart drinking straw tip assembly (100) and pressure acting on the surface of the liquid.

FIG. 5 shows different orientations of the smart drinking straw tip assembly (100)

FIG. 6 shows how the non-contact liquid detection sensor is used to calculate users' actual consumption.

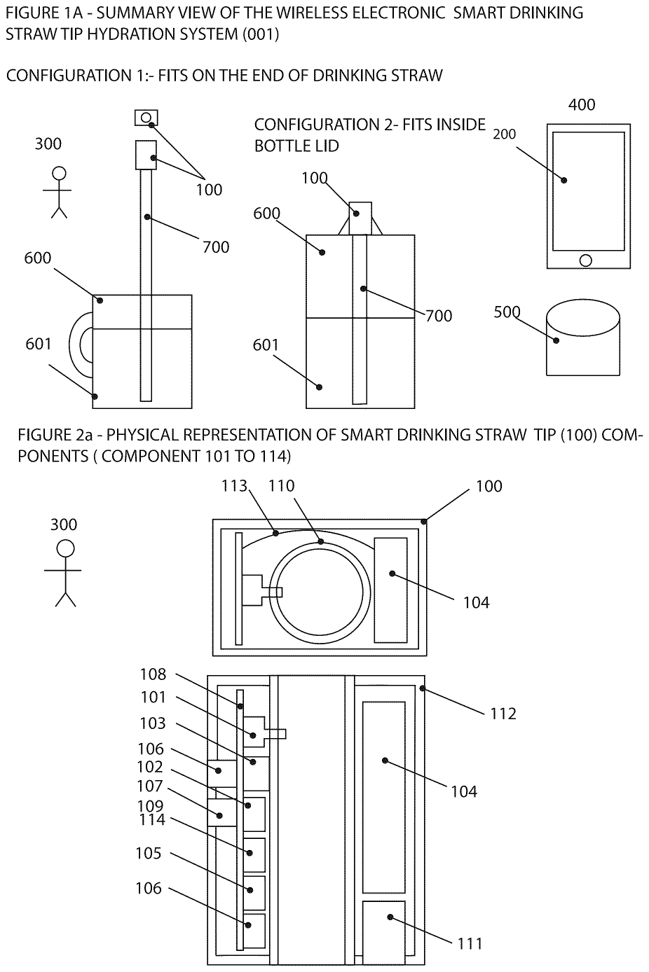

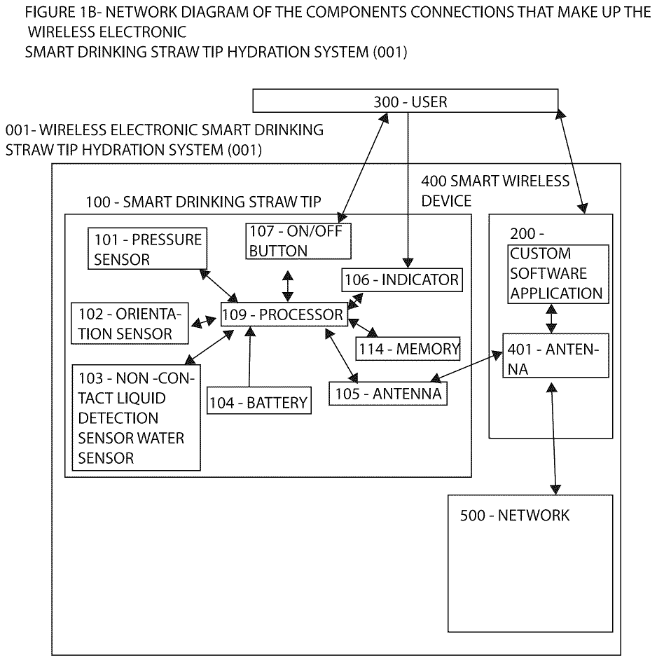

FIG. 1A shows the invented components and support components of the wireless electronic smart drinking straw tip system (001). The invented components are what the patent is protecting. The invented components of the system are smart drinking straw tip assembly (100) and custom software application (200). The support components of the system are user (300), wireless device (400), network (500), liquid container (600), liquid (601) and drinking straw (700).

The smart drinking straw tip assembly (100) is fitted on the top end of the drinking straw (700), the bottom end of the drinking straw is inside the liquid container (600) that's filled with liquid (601). The user (300) sucks on the smart drinking straw tip assembly (100) creating a pressure vacuum inside the smart drinking straw tip assembly (100) and drinking straw (700) causing liquid (601) to raise up the drinking straw (700) and smart drinking straw tip assembly (100) and the user (300) consumes the liquid (601).

The user (300) enters the users details (301) into the custom software application (200).

Network (500) communicates with wireless device (400) and stores consumption data and user details (201). Liquid container (600) contains the liquid (601) consumed by the user (300). liquid (601) is consumed by the user (300) though the drinking straw (700) and smart drinking straw tip assembly (100). The smart drinking straw tip assembly (100) calculates the users' actual liquid consumption (602) and sends the amount consumed to the wireless device (400), the wireless device (400) passes the consumed amount to the custom software application (200). The user (300) can see how much they have consumed on the custom software application (200). The custom software application (200) then compares the users actual liquid consumption (602) via the smart drinking straw tip assembly (100) to the total adjusted recommended consumption (210), if above or below the custom software application (200) sends a signal using wireless technology back to the smart drinking straw tip assembly (100) which triggers an audio or visual indicator on the smart drinking straw tip assembly indicator (106) to indicate to user (300).

Configuration 1, shows the smart drinking straw tip assembly (100) on the end of a drinking straw (700). Configuration 2, shows the smart drinking straw tip assembly (100) inside the bottle lid.

FIG. 1B shows network diagram of the component connections for the smart drinking straw tip hydration system (001). Once the device is turn on by the user (300) using the on/off button (107) the processor (109) polls the pressure sensor (101), orientation sensor (102) and non-contact liquid detection sensor (103) for values, these values are stored in memory (114) and then used by the processor (109) to calculate the users actual liquid consumption (602). The processor (109) then sends values to the wireless antenna (105) which transmits the values to the wireless smart device antenna (401). There are different technologies for wireless data transfer most notably the Bluetooth technology.

Battery (104) is to power the other components in the smart drinking straw tip assembly (100) and is connected via the on/off button (107).

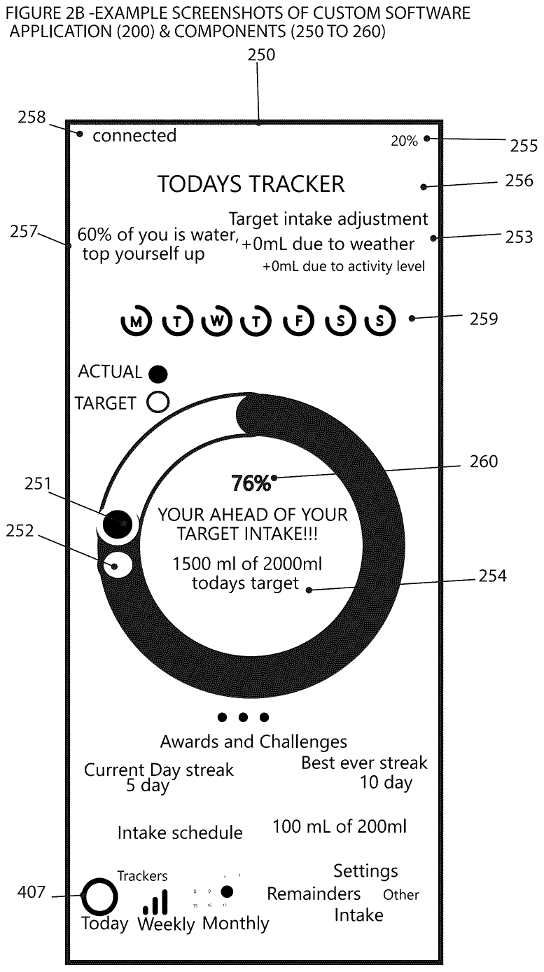

FIG. 2A shows the physical representation of the smart drinking straw tip assembly (100) and components (101 to 114). The smart drinking straw tip assembly (100) is turned on by the user (300), using the Power on/off button (107). The battery (104) which is connected to the other components via the power flexi printed circuit (113), powers all electronic components. The tube (110) is where the liquid (601) flows to the user (300). There is a small hole in the tube (110) for the nozzle of the pressure sensor (101) so it can measure the pressure inside the tube (110). The orientation sensor (102) is placed on the outside of the tube (110) to enable it to measure the orientation of the tube (110) and therefore the smart drinking straw tip assembly (100). The non-contact liquid detection sensor (103) is placed on the outside of the tube (110) to enable it to detect liquid (601), on the inside of the tube (110). The processor (109) polls the three sensors (101, 102, 103) for measurements and calculates users actual liquid consumption (602) using the measurements. Once the consumption is calculated the processor (109) via the antenna (105) sends the users actual liquid consumption (602) to the wireless device (200). The indicator (106) is used to indicate to the user (300) when they are below the total adjusted recommended consumption (210). The connector (111) connects the battery (104) to a power source to recharge the battery (104). The battery (104) can also be recharged wirelessly using wireless charging technologies such as QI and NFC. The enclosure (112) encloses and protects all the electronic components of the smart drinking straw tip assembly (100). Electronic components are electrically connected by the printed circuit board (108) and Power flexi printed circuit (113).

FIG. 2B shows example screenshot of customer software application (200), Todays tracker page (250). The todays tracker page (250) is the home page of the custom software application (200). It shows the Users actual consumption indicator (251) against the users target consumption indicator (252). There is also text stating actual consumption vs target consumption (254) and Percentage of actuals consumption compared to target recommended consumption (260). The adjustments made to baseline target consumption (253) are shown and split by weather and activity level. Remaining battery life of straw tip (255) and connection status (258) between smart drinking straw tip assembly (100) and wireless device (200). Tittle of current page selected (256), Motivational message (257) and previous days consumption summary (259) are included on the page. There are also weekly and monthly pages.

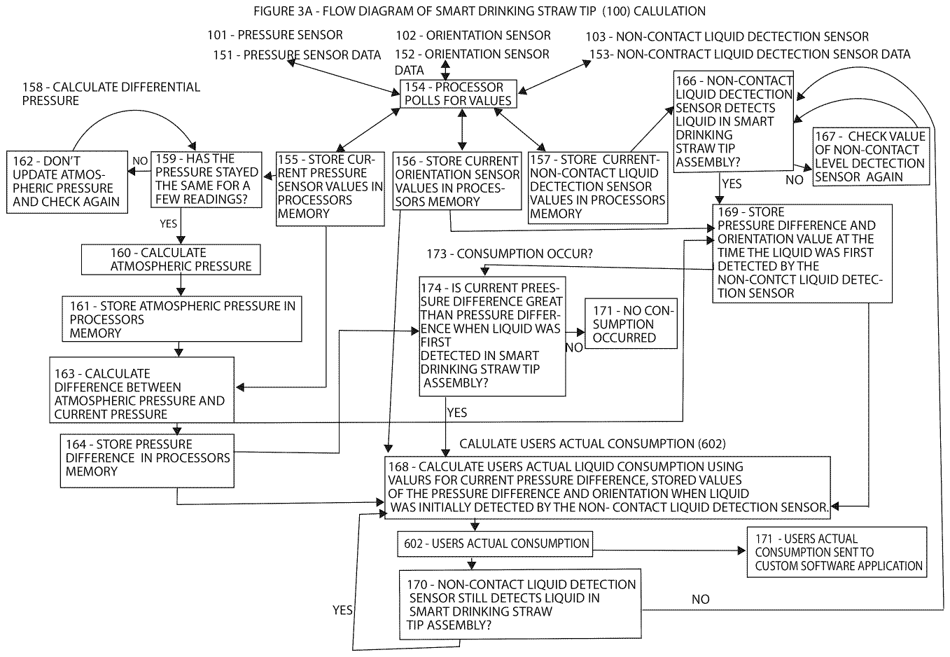

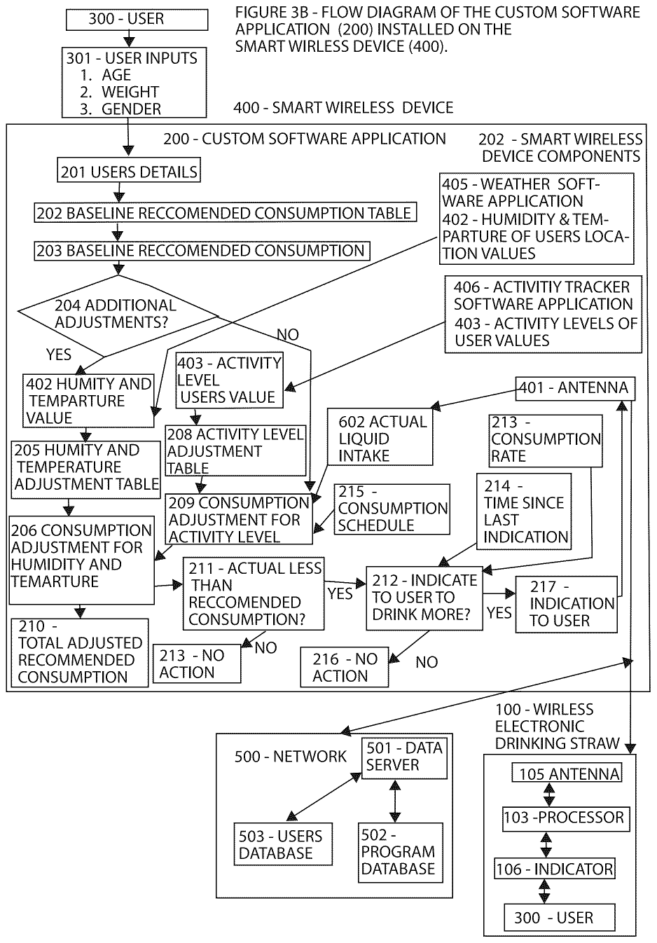

FIG. 3A shows a flow diagram for the smart drinking straw tip assembly (100) calculations

After the smart drinking straw tip assembly (100) is turned on the processors polls for values (154) of the three sensors (101,102,103). These values are then individually stored in the processors memory, store pressure sensor values (155), store orientation sensor values (156) and store and non-contact liquid detection sensor values (157).

158—calculate differential pressure—To calculate pressure differences between the pressure on the surface of the liquid in the container (atmospheric pressure) and pressure inside the smart drinking straw tip assembly (current pressure reading) we first calculate atmospheric pressure. Atmospheric pressure is calculated when the smart drinking straw tip assembly (100) is not being sucked, this is determined by having consecutive pressure readings that are the same. Has the pressure stayed the same for a few readings (159)? if no atmospheric pressure isn't updated. If yes then calculate the atmospheric pressure (160), this is the average of the pressure readings. The atmospheric pressure value is then stored in processors memory (161) so it can be used to calculate the pressure difference between atmospheric pressure and current pressure. If the smart drinking straw tip assembly (100) is not sucked there will be no difference.

Then calculate (163) difference between atmospheric pressure (161) and current pressure (155), this value is then stored in the processor's memory (164).

Check if the non-contact liquid detection sensor detects liquid in the smart drinking straw tip assembly (166). If yes then the pressure and orientation values are stored at the time the liquid was initially detected by the non-contact liquid detection sensor (167).

174—Consumption only occurs when the current pressure difference (164) is greater than the pressure difference to get the liquid up to the level of the non-contact liquid detection sensor (169).

If the pressure difference is lower, no consumption will occur (171) if pressure difference is higher the user's actual consumption (602) is calculated.

168—Values for the current pressure difference (164), current orientation value (156), stored values of the pressure difference and orientation at the time the liquid was initially detected by the non-contact liquid detection sensor (167) are used to calculate users' actual consumption (602).

The users actual consumption is continuous calculated until the non-contact liquid detection sensor stops detecting liquid (170).

175—The calculated users actual consumption (602) is sent to the custom software application (200).

FIG. 3B shows a flow diagram of the custom software application (200) installed on a smart wireless device (400).

How much liquid a user needs to consume is dependent on many factors including age, gender, weight, temperature/humidity of users location and users level of activity. Custom software application (200) calculates total adjusted recommended consumption (210) using users details (201) gender, age, weight and temperature/humidity of users location (402) and users level of activity (403).

The user (300) inputs values for the users details (201), age, weight and gender into the custom software application (200), the application then looks up the recommended consumption table (202) and using the users details (201) looks up a baseline recommended consumption amount (203). For example, a female, aged 25, weighting 10 stone recommended baseline consumption (203) is 2000 ml per day.

The custom software application (200) has two optional adjustments to the baseline recommended consumption (203) that the user (300) can enable. These are for the users level of activity (403) and Humidity & temperature of users location (402). The users level of activity (403) is taken from an activity tracker software application (406) that is already installed on the wireless device (400). The user (300) selects which activity tracker software application (406) to get the data from. The Humidity & temperature of users location (402) is taken from weather software application (405) that is already installed on the wireless device (400). The user (300) selects which weather software application (405) to get the data from.

Using the humidity and temperature values (402) the custom software application (200) looks up the humidity and temperature adjustment table (205) to see how much to adjust the baseline recommended consumption (203).

Using the activity level value (403) the custom software application (200) looks up activity level adjustment table (208) to see how much to adjust the baseline recommended consumption (203).

The baseline recommended consumption (203) is adjusted by consumption adjustment for humidity/temperature (206) and consumption adjustment for activity level (209) to total adjusted recommended consumption (210).

The custom software application (200) compares users actual liquid consumption (602) through the smart drinking straw tip assembly (100) to the total adjusted recommend target consumption (210).

211—If the users actual consumption (602) is less than total adjusted recommend consumption (210) and below consumption schedule (215) go to next stage (212) Indicate to user to drink. The indicate to user to drink more (212) looks at the current consumption rate (213) and time since last indication (214) to determine in indication should be sent to user. If yes then the custom software application (200) will indicate to user (217) by sending a signal via the wireless devices antenna (401) to the straws antenna (105), the signal is then processed by the processor (109) which turns on the straws indicator (106) to inform the user (300) that consumption rate is below target and prompting the user (300) to drink.

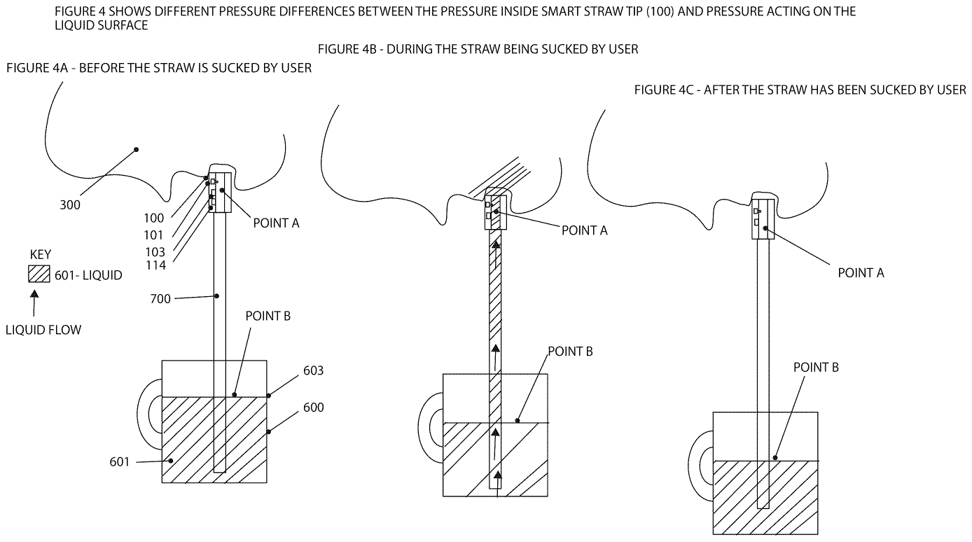

FIG. 4 shows different pressure differences between inside smart drinking straw tip assembly (Point A) and on the surface of the liquid (Point B).

FIGS. 4a, 4b & 4c demonstrates how pressure plays a key role in users consumption and how one pressure sensor can be used to calculate the differences at two locations (point A and B).

At Point A the pressure sensor (101) inside the smart drinking straw tip assembly (100) takes pressure readings, if the pressure remains the same for a few readings then that is used for the value of atmospheric pressure and is stored in the processors memory (114). As point B is always at atmospheric pressure, the processor then uses the atmospheric pressure value to calculate the pressure differences between point A and B. When the smart drinking straw tip assembly (100) is sucked the pressure at point A changes and so the difference between point A and point B can be calculated.

FIG. 4a shows the liquid (601) and pressure points (A & B) before the smart drinking straw tip assembly (100) is sucked by the user (300). Before the user (300) sucks the smart drinking straw (100) tip the pressure at Point A and point B are both at atmosphere pressure (e.g) 1000 hectoPascals (hPa). Points A & B are both at the same pressure (atmospheric pressure) and therefore liquid (601) remains in the container (600).

FIG. 4b shows the liquid (601) and pressure points (A & B) during the smart drinking straw tip assembly (100) being sucked by the user (300).

When the smart drinking straw tip assembly (100) is sucked it decreases the pressure at Point A by creating a small vacuum (e.g) 15-40 hPA below atmospheric pressure which creates a pressure difference between point A and B. The atmospheric pressure on the surface of the liquid at Point B is higher than the pressure inside the smart drinking straw tip assembly (100) at Point A. This results in the atmospheric pressure forcing the liquid (601) up the straw (700) into the lower pressure area through the drinking straw (700) and smart straw tip (100) and into the users (300) mouth.

FIG. 4c shows the liquid (601) and pressure levels after the smart drinking straw tip assembly (100) has been sucked by the user (300). Once the user (300) stops sucking the smart drinking straw tip assembly (100), Point A increases in pressure to the same pressure as Point B (atmospheric pressure). As Point A and Point B are now the same pressure any remaining liquid (601) inside the straw (700) is pulled by back into the liquid container (600) by gravity. The state then goes back to FIG. 4a.

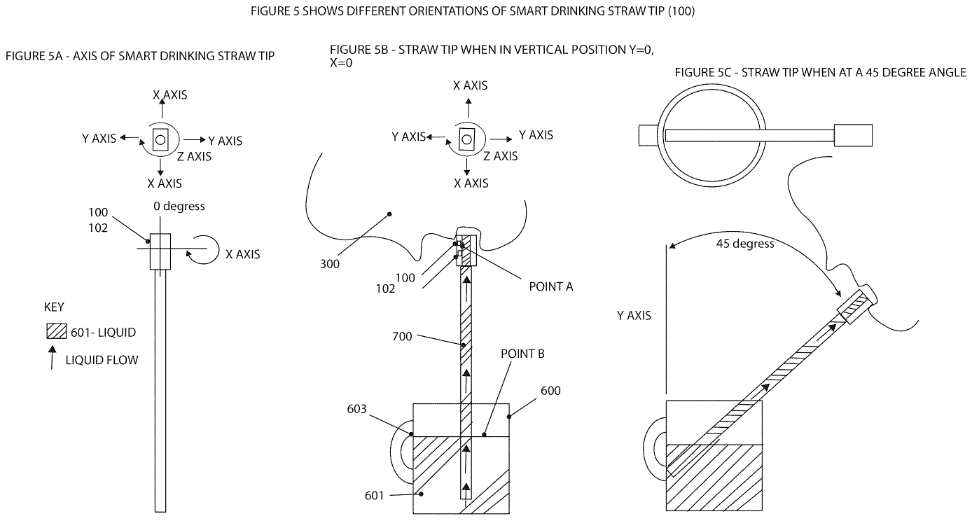

FIG. 5 shows different orientations of the smart drinking straw tip assembly (100). FIG. 5 explains how the orientation of the smart drinking straw tip assembly (100) and straw (700) plays a role in determining how much liquid (601) is consumed at a given pressure difference between Point A and Point B. The higher the angle from 0 degree in either the X or Y axis the more liquid (601) will be consumed at a given pressure difference and distance (between Point A & B).

FIG. 5a shows the axis orientation of the smart drinking straw tip assembly (100), the orientation is obtained from an orientation sensor (102) inside the smart drinking straw tip assembly (100).

FIG. 5b shows the smart drinking straw tip assembly (100) in an upright (horizontal) position x=0 degrees, Y=0 degrees orientation, at this orientation the pressure difference between Point A and Point B, needed for liquid to flow up the straw (700) is the highest because of gravity and liquid flow.

When the straw is in the upright position user's actual consumption can be accurately estimated without the orientation sensor data. Orientation sensor data is needed to increase accuracy when the straw is not upright (at an angle).

FIG. 5c shows the smart drinking straw (100) tip rotated around the Y axis at 45 degrees away from centre line, X axis at 0 degrees away from the centre line. Compared to FIG. 5b a lower pressure difference (between Points A and B) is needed for liquid to flow up the straw (700) for a given distance between Point A and B.

The larger the rotation away from the centre line, the less pressure is needed for consumption to occur. For the users actual liquid consumption (602) calculation, X and Y axis have the same impact.

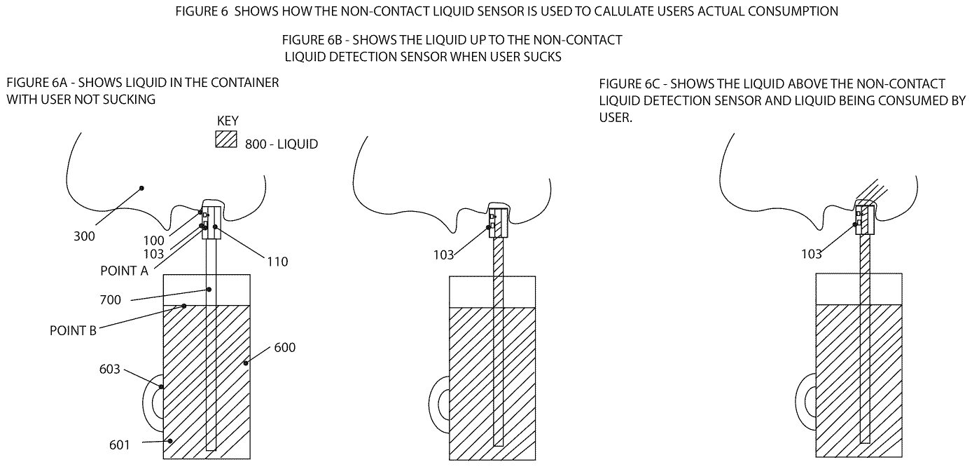

FIG. 6 shows how the non-contact liquid sensor is used to calculate users actual consumption.

A non-contact liquid detection sensor (103) can detect when liquid (601) has made it inside the smart drinking straw tip assembly tube (110). By calculating the pressure difference between Points A and B, and orientation when the non-contact liquid detection sensor (103) initially detected liquid inside the smart straw tip tube (110) and knowing current pressure difference between Point A and B and orientation the user actual consumption (602) can be calculated.

FIG. 6a shows liquid in the container when the smart straw tip assembly (100) is not sucked by user (300). The pressure at points A and B are both at atmospheric pressure (e.g) 1000 hPa

FIG. 6b shows the liquid up to the level of the non-contact liquid detection sensor when the user starts sucking. The non-contact liquid detection sensor detects liquid and the pressure difference is stored (e.g) 980 hPa (20 hPa difference).

FIG. 6c shows the liquid above the non-contact liquid detection sensor and liquid consumption is occurring. The user has increased the pressure difference between Points A and B resulting in liquid consumption. (e.g) 950 hPa (50 hpa difference). This is 30 hPa more than when liquid reached the non-contact liquid detection sensor.

Component table

| Figure | |
|---|---|
| reference | |
| number | Component |
| 001 | wireless electronic smart drinking straw tip |
| | hydration system (001) |
| 100 | smart drinking straw tip assembly |
| 101 | Pressure sensor |
| 102 | orientation sensor |
| 103 | non-contact liquid detection capacitive sensor |
| 104 | battery |
| 105 | antenna |
| 106 | indicator |
| 107 | Power ON/OFF button |
| 108 | printed circuit board |
| 109 | processor |
| 110 | tube |
| 111 | power connector |
| 112 | enclosure |
| 113 | Power flexi printed circuit board |
| 114 | processors memory |
| 151 | Pressure sensor data |
| 152 | Orientation sensor data |
| 153 | Non-contact liquid detection sensor data |
| 154 | Processor Polls for values |
| 155 | Store pressure sensor values in processor memory |
| 156 | Store orientation sensor values in processor memory |
| 157 | Non-contact liquid detection sensor values in |
| | processor memory |
| 158 | Calculate differential pressure |
| 159 | Has the pressure stayed the same for a few readings |
| 160 | Calculate atmospheric pressure |
| 161 | Store atmospheric pressure in processors memory |
| 162 | Don't update atmospheric pressure and check again |
| 163 | Calculate difference between atmospheric pressure |
| | and current pressure |
| 164 | Store pressure difference in processors memory |
| 166 | Non-contact detection sensor detects liquid in |
| | smart drinking straw tip assembly? |
| 167 | Check value of Non-contact detection sensor again |
| 168 | Analyse and calculate users actual consumption (602) |
| | using difference in pressure, orientation and distances |
| | between smart drinking straw tip and liquid level |
| | in container. |
| 169 | Store pressure difference and orientation values at the |
| | time liquid was detected by the non-contact liquid |
| | detection sensor |
| 170 | Non-contact liquid sensor still detects liquid in smart |
| | drinking straw tip assembly? |
| 171 | No consumption occurred |
| 174 | Is pressure difference greater than pressure difference |
| | when liquid was initially detected in the smart drinking |
| | straw tip assembly? |
| 175 | The calculated users actual consumption (602) is sent |
| | to the custom software application (200). |
| 200 | custom software application |
| 201 | Users details |
| 202 | Recommended consumption table |
| 203 | Baseline recommended consumption |
| 204 | Additional adjustments |
| 205 | Humidity and temperature adjustment table |
| 206 | Consumption adjustment for Humidity and temperature |
| 207 | |
| 208 | Activity level adjustment table |
| 209 | Consumption adjustment for activity level |
| 210 | Total adjusted recommended consumption |
| 211 | Actual less than recommended consumption? |
| 212 | Indicator to User |
| 213 | No action |
| 214 | Time since last indication |
| 215 | Consumption schedule |
| 216 | No action |
| 217 | Indication to user |
| 250 | custom software application - Todays tracker page |
| 251 | Users actual consumption indicator |
| 252 | Users target consumption indicator |
| 253 | Adjustments made to baseline target consumption |
| 254 | Text stating actual consumption vs target consumption |
| 255 | Remaining battery life of straw tip |
| 256 | Tittle of current page selected |
| 257 | Motivational message |
| 258 | Connection status between drinking straw tip and |
| | wireless device (custom software application |
| 259 | Previous days consumption summary |
| 260 | Percentage of actuals consumption compared to |
| | target recommended consumption |
| 300 | User |
| 301 | User inputs |
| 400 | Smart wireless device (e.g) smart phone, |
| | smart tablet |
| 401 | Smart wireless device antenna |
| 402 | Smart wireless device humidity and |
| | temperature values |
| 403 | Smart wireless device activity level values |
| 404 | Smart wireless device memory |
| 405 | Weather software application |
| 406 | Activity level tracker software application |
| 500 | Network |
| 501 | Data server |
| 502 | Program database |
| 503 | Users database |
| 600 | Liquid container |
| 601 | Liquid |
| 602 | Users actual liquid consumption |
| 603 | Liquid level inside container |
| 700 | Drinking straw |

1. A wireless electronic smart drinking straw tip hydration system comprising;
(A) a smart drinking straw tip assembly to calculate users actual liquid consumption comprising;
a hollow tube that fits on top of a drinking straw of which the drinking straws bottom is in a liquid container;
a pressure sensor, to measure pressure inside the hollow tube;
a orientation sensor for detecting spatial orientation of the smart drinking straw tip assembly;
a non-contact liquid detection sensor for detecting when liquid has reached inside the smart drinking straw tip assembly hollow tube;
a processor, operably coupled to the pressure sensor, non-contact liquid detection sensor and orientation sensor, to poll and store measurements of the sensors and based on the measurements,
calculate atmospheric pressure using pressure sensor measurements,
calculate pressure differences between atmospheric pressure and current pressure using pressure sensor measurements,
calculate the users actual liquid consumption through the smart drinking straw tip assembly by using
1) calculated pressure difference and orientation sensor values when non-contact liquid detection sensor initially detects liquid inside the smart drinking straw tip assembly hollow tube,
2) calculated current pressure differences and orientation sensor values while the non-contact liquid detection sensor still detects liquid inside the hollow tube;
a antenna couped to the processor to transmit data such as users actual liquid consumption to the custom software application installed on the wireless device (e.g) smart phone and to receive data from custom software application via wireless device to turn on indication to user;
a indicator operably coupled to a processor to provide indication to user if they are above or below users recommended scheduled target liquid consumption and prompt user to drink more or less;
(B) custom software application installed on a wireless device, receives users actual liquid consumption from smart drinking straw tip assembly via wireless device and displays users actual consumption values to user, calculates users recommended scheduled target liquid consumption based on users inputs and additional optional adjustments and displays these values to user, compares users actual liquid consumption against users recommended scheduled target liquid consumption and displays differences to user and sends data back to smart drinking straw tip assembly via wireless device to indicate to user if above or below users recommended scheduled target liquid consumption.

2. The system of claim 1, wherein the pressure sensor is polled by the processor to take pressure measurements, if pressure remains unchanged for a few measurements, the processor then calculates atmospheric pressure by taking the average of those measurements and stores the value for atmospheric pressure.

3. The system of claim 2, the current pressure difference is then continuously calculated by the processor by subtracting current pressure measurement from calculated atmospheric pressure and stores the value.

4. The system of claim 1, the calculated pressure difference and orientation measurement when the non-contact liquid detection sensor initially detects liquid inside the smart drinking straw tip assembly hollow tube are stored by the processor.

5. The system of claim 1, wherein a orientation sensor comprises a plurality of accelerometer, gyroscope and magnetometer sensor configured to detect spatial orientation of the smart drinking straw tip assembly in three spatial dimensions.

6. The system of claim 1, wherein a non-contact detection sensor is fitted on the outside of the smart drinking straw tip assembly hollow tube and detects liquid inside smart drinking straw tip assembly hollow tube via change in capacitance or resistance.

7. The system of claim 1, the custom software application displays users actual liquid consumption through the smart drinking straw tip assembly and recommended scheduled target liquid consumption to the user and shows actual liquid consumption versus users recommended scheduled target liquid consumption.

8. The system of claim 1, the custom software application produces a users recommended scheduled target liquid consumption based on users inputs such as age, gender and weight.

9. The system of claim 8, the custom software application has additional optional adjustments to the users recommended scheduled target liquid consumption which adjusts for temperature and humidity based on the geographical location of the wireless device that the custom software application is installed on.

10. The system of claim 8, the custom software application has additional optional adjustments to the users recommended scheduled target liquid consumption which adjusts for users level of activity based on user selected fitness tracker application data installed on the wireless device.

11. The system of claim 1, the custom software application when deciding if to send data to the smart drinking straw tip assembly to indicate to user primarily compares users recommended scheduled target liquid consumption against users actual liquid consumption through smart drinking straw tip assembly and secondarily considers current liquid consumption rate and time since last data and indication sent.
